(19) United States
(12) Patent Application Publication
(10) Pub. No.: US 2021/0361969 A1
(43) Pub. Date: Nov. 25, 2021

(54) SYSTEMS AND METHODS FOR THERAPY DELIVERY USING NEAR FIELD MAGNETIC INDUCTION DEVICES

(71) Applicant: NearField Atomics Inc., Seattle, WA (US)

(72) Inventor: John T. Butters, Seattle, WA (US)

(21) Appl. No.: 17/320,144

(22) Filed: May 13, 2021

Related U.S. Application Data
(60) Provisional application No. 63/029,170, filed on May 22, 2020.

(51) Int. Cl. A61N 2/02; G16H 40/67; G16H 20/30

(52) U.S. Cl. CPC A61N 2/02; G16H 20/30; G16H 40/67

(57) ABSTRACT

A therapy delivery system includes a near field magnetic induction (NFMI) transceiver device configured to be worn or otherwise disposed on a patient, the NFMI transceiver device including a NFMI transceiver; and a therapy delivery device including a NFMI receiver configured to receive signals from the NFMI transceiver device and a therapy delivery circuit configured to deliver a therapeutic magnetic signal, based, at least in part, on the received signals from the NFMI transceiver device, to the patient when the patient wears the therapy delivery device or has the therapy delivery device disposed on skin of the patient or has the therapy delivery device implanted.

### CROSS-REFERENCE TO RELATED APPLICATIONS

This patent application claims priority to U.S. Provisional Application Ser. No. 63/029,170, filed May 22, 2020, which is incorporated herein by reference.

### FIELD

The present invention is directed to the area of systems and methods for therapy delivery and control of therapy delivery. The present invention is also directed to systems and methods for therapy delivery and control of therapy delivery using near field magnetic induction for communication between devices.

### BACKGROUND

A variety of therapy delivery devices and sensors have been developed including devices or sensors that are implanted, disposed on the skin of the patient, or worn by the patient. In some cases, the therapeutic devices or sensors communicate with a device to receive instructions or deliver data. In addition, the therapy delivery devices or sensors may receive power from a power source to continue operation.

### BRIEF SUMMARY

One embodiment is a therapy delivery system that includes a near field magnetic induction (NFMI) transceiver device configured to be worn or otherwise disposed on a patient, the NFMI transceiver device including a NFMI transceiver; and a therapy delivery device including a NFMI receiver configured to receive signals from the NFMI transceiver device and a therapy delivery circuit configured to deliver a therapeutic magnetic signal, based, at least in part, on the received signals from the NFMI transceiver device, to the patient when the patient wears the therapy delivery device or has the therapy delivery device disposed on skin of the patient or has the therapy delivery device implanted.

In at least some embodiments, the therapy delivery device further includes a power source coupled to the NFMI receiver and the therapy delivery circuit. In at least some embodiments, the therapy delivery device further includes a charging circuit coupled to the power source and configured to receive power from an external source to charge the power source. In at least some embodiments, the NFMI transceiver device includes a charging circuit configured to provide power to the charging circuit of the therapy delivery device. In at least some embodiments, the charging circuit of the NFMI transceiver device is configured to utilize the NFMI transceiver to provide the power to the charging circuit of the therapy delivery device.

In at least some embodiments, the therapy delivery circuit is configured to deliver the therapeutic magnetic signal having a frequency in a range of 0.001 Hz to 22 kHz. In at least some embodiments, the therapy delivery device further includes a first substrate and the therapy delivery circuit and the NFMI receiver are disposed on the first substrate. In at least some embodiments, the therapy delivery device further includes an adhesive disposed on a surface of the substrate for adhering the therapy delivery device to the skin of the patient. In at least some embodiments, the therapy delivery device includes a second substrate disposed over the therapy delivery circuit and the NFMI receiver and attached to the first substrate.

In at least some embodiments, the therapy delivery system further includes a sensor device including a sensor circuit configured to produce a sensor signal based on observation of the patient by the sensor device; and a NFMI transmitter configured to transmit the sensor signal to the NFMI transceiver device. In at least some embodiments, at least one of the therapy delivery device or the sensor device is implantable in the patient.

In at least some embodiments, the NFMI transceiver device further includes a communications circuit for communication, other than NFMI, to a user device. In at least some embodiments, the therapy delivery system further includes the user device configured to communicate with the NFMI transceiver device through the communications circuit of the NFMI transceiver device. In at least some embodiments, the user device includes a mobile phone, a tablet, or a computer. In at least some embodiments, the NFMI transceiver device further includes a processor coupled the NFMI transceiver and a memory coupled to the processor.

Another embodiment is a method of delivering therapy to patient that includes receiving, at a therapy delivery device, a near field magnetic induction (NFMI) signal from a NFMI transceiver device worn or otherwise disposed on the patient and, in response to the received NFMI signal, delivering a therapeutic magnetic signal to the patient using the therapy delivery device, wherein the therapy delivery device is worn by the patient, disposed on skin of the patient, or implanted in the patient.

In at least some embodiments, the method further includes receiving, at the therapy delivery device, a charging signal from the NFMI transceiver device to charge a power source of the therapy delivery device. In at least some embodiments, receiving the charging signal includes receiving the charging signal using NFMI.

In at least some embodiments, the method further includes receiving, at the NFMI transceiver device, a NFMI signal from a sensor device that is worn by the patient, disposed on skin of the patient, or implanted in the patient. In at least some embodiments, the method further includes communicating, from the NFMI transceiver device without using NFMI, with a user device.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a block diagram of one embodiment of a system for delivering therapy using devices that communicate through near field magnetic induction (NFMI), according to the invention;

FIG. 2 illustrates one embodiment of a positioning of a NFMI transceiver device and a therapy delivery device on the body of a patient, according to the invention;

FIG. 3 is a schematic diagram of one embodiment of NFMI communication, according to the invention;

FIG. 4 is a block diagram of one embodiment of system components including a NFMI transceiver device, a therapy delivery device, and a sensor device, according to the invention;

FIG. 5A is a schematic diagram of one embodiment of a therapy delivery device using NFMI communication, according to the invention;

FIG. 5B is a schematic cross-sectional view of a portion of the therapy delivery device of FIG. 5A, according to the invention; and

FIG. 6 is a schematic front view of one embodiment of a NFMI transceiver device, according to the invention.

### DETAILED DESCRIPTION

The present invention is directed to the area of systems and methods for therapy delivery and control of therapy delivery. The present invention is also directed to systems and methods for therapy delivery and control of therapy delivery using near field magnetic induction for communication between devices.

The development of new sensors and therapy delivery devices, as well as miniaturization of previously developed sensors and therapy delivery devices, provides an opportunity for monitoring of health conditions of patients. Such monitoring may be continuous or periodic and may be available at home or elsewhere, instead of being relegated to a healthcare facility. The use of wireless connectivity technologies can facilitate operation of, and data collection from, sensors and therapy delivery devices. The creation of a single body central gateway, such as a Wireless Body Area Network (WBAN), to transmit or receive from one or more therapy delivery devices or sensors can enhance this operation and data collection. The use of a WBAN can facilitate the collection of data for patient treatment of diseases or disorders, such as, for example, chronic diseases, like diabetes mellitus, cardiovascular diseases, respiratory diseases, cancer, other serious diseases, or the like. In at least some embodiments, data can be further exchanged between the patient and a healthcare provider (for example, a doctor, surgeon, clinic, hospital, or the like or any combination thereof). Such exchanges may facilitate mobile health (mHealth) or Telehealth services and applications.

Near Field Magnetic Induction (NFMI) is a short-range wireless communication technology that utilizes magnetic fields for inductive transmission between coils or transducers in individual devices, in contrast to many conventional communications techniques that utilize electrical transmission through antennas. In at least some embodiments, NFMI can be superior to electrical/antenna transmission technologies for body-area networks, such as WBAN, because NFMI is attenuated less by the body than many electrical/antenna transmission technologies. Also, NFMI signals are attenuated more strongly over distance (in at least some cases, approximately by a factor of 1/r⁶ where r is distance) than a number of other conventional transmission technologies and, therefore, NFMI may provide a more private network that is substantially limited to the body of the patient. This can reduce interference or privacy breaches. In at least some embodiments, NFMI is more power efficient than other wireless technologies such as Bluetooth™, near field communication (NFC), or the like and may have lower power consumption than these technologies for transmitting the same data or signals. In addition to communication using NFMI, in at least some embodiments, the same coils can be used for wireless charging of device batteries or other power sources using magnetic induction.

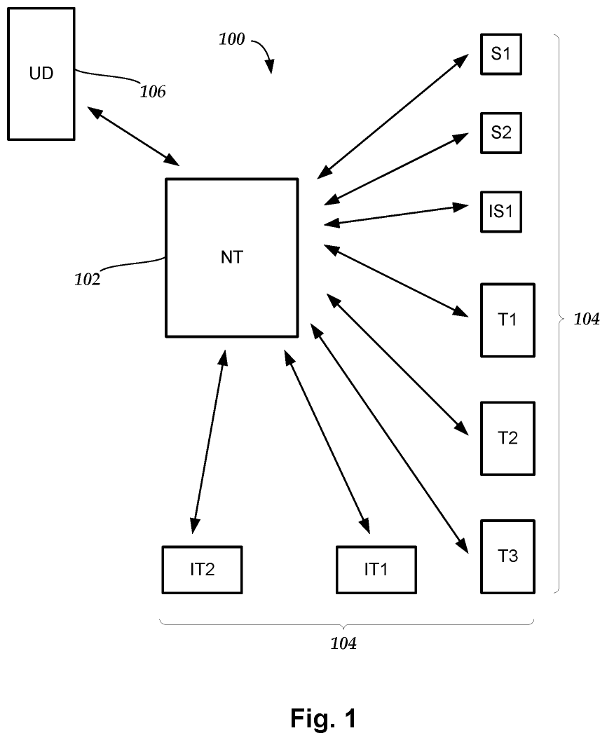

As described herein, a therapy delivery system can utilize NFMI to communicate with, and optionally control, one or more therapy delivery devices or sensors or any combination thereof. FIG. 1 illustrates one embodiment of a therapy delivery system 100 that includes a NFMI transceiver device 102 (NT) and one or more system devices 104 that can be therapy delivery devices (for example, devices T1, T2, T3, IT1, and IT2), sensors (for example, sensors S1, S2, and IS1), or the like or any combination thereof. Examples of sensors include, but are not limited to, temperature sensors such as thermistors or infrared sensors; piezoelectric or other pressure sensors (to measure, for example, blood pressure, pulse rate, inhalation/expiration, or other physical parameters); fluid sensors such as sweat sensors or blood sensors (for example, glucose sensors); pH sensors; cameras; microphones; healing detection sensors; or the like or any combination thereof.

The therapy delivery system 100 can optionally include one or more user devices 106 (UD) which may be, for example, a mobile device (such as a mobile phone, tablet, laptop, personal data assistant, or the like), a computer (for example, a laptop, a desktop computer, a server, or the like), a dedicated programming or monitoring device, or the like or any combination thereof. A user device 106 can, for example, direct operation of, or control, the NFMI transceiver device 102 or one or more of the system device 104 or any combination thereof; program the NFMI transceiver device 102 or one or more of the system device 104 or any combination thereof; process or analyze data from any of the system devices 104; transmit data or other information to other devices, such as a computer or server at a healthcare facility; provide information to a user or patient on a screen of the user device 106; or the like or any combination thereof. In at least some embodiments, the delivery of therapy or the programming or alteration of therapy parameters may be restricted to a user with credentials, such as a password or other identification. The user device 106 may include one or more programs, applications, or features that provide these functions. In at least some embodiments, the NFMI transceiver device 102 can also perform one or more of these functions of a user device 106.

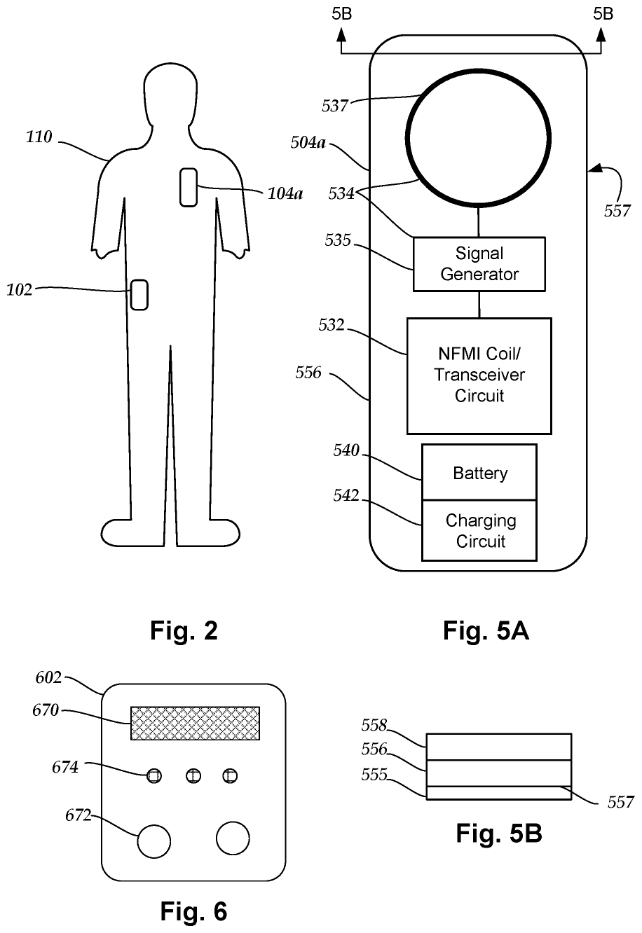

The NFMI transceiver device 102 communicates with the one or more system devices 104 by NFMI. In at least some embodiments, the NFMI transceiver device 102 and one or more system devices 104 create a Wireless Body Area Network (WBAN) with NFMI transmission. In at least some embodiments, the NFMI transceiver device 102 can be worn or carried by the patient. Each of the system devices 104 can be independently disposed on the patient, worn by the patient, or implanted in the patient. Devices 104 labeled IS1 and IT1/IT2 are an implanted sensor and implanted therapy devices, respectively. FIG. 2 illustrates one example of a NFMI transceiver device 102 worn by the patient 110 and a therapy delivery device 104a positioned on the patient.

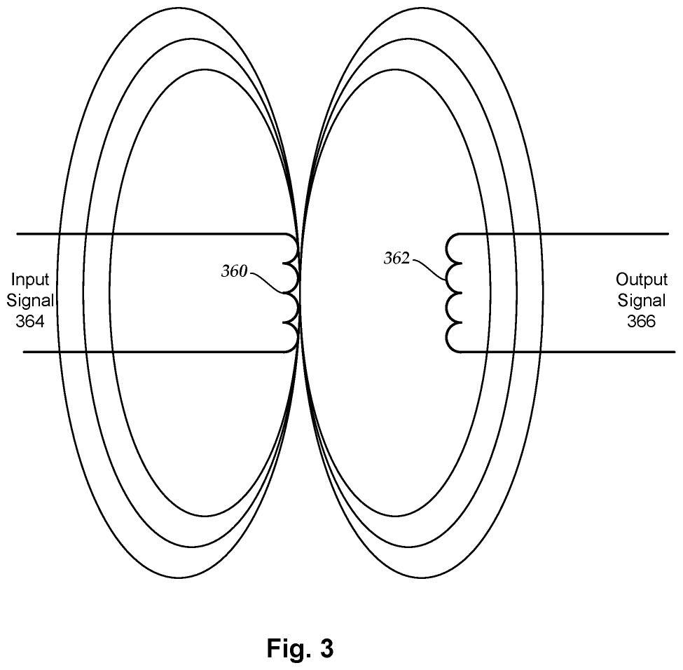

FIG. 3 schematically illustrates NFMI transmission of an input signal 364 by magnetic field induction from a transmitting coil 360 (for example, in a NFMI transceiver 102) to a receiving coil 362 (for example, in a system device 104) to produce an output signal 366. As an alternative to the transmitting coil 360 or receiving coil 362 and other suitable transducer can be used that facilitates NFMI transmission/reception. The use of the term “coil” herein includes other suitable transducers unless indicated otherwise.

NFMI can use any type of analog or digital modulation for signal transmission including, but not limited to, any type of amplitude, frequency, phase, or other modulation. In at least some embodiments, the same coils 360, 362 can be used for transferring power to a device by magnetic induction. In other embodiments, different coils or antennas may be used for transferring power to the device. In at least some embodiments, the transmitting coil 360 and receiving coil 362, and associated circuitry, can both transmit and receive so that the NFMI transceiver device 102 and system devices 104 can communicate in both directions using NFMI.

Returning to FIG. 1, in at least some embodiments, the NFMI transceiver device 102 can communicate with one or more user devices 106 using any suitable communications arrangement or protocol including, but not limited to, NFMI, Bluetooth™, near field communications (NFC), wireless fidelity (WiFi), satellite communication, cellular communication, Infrared Data Association standard (IrDA), or the like or any combination thereof. In some embodiments, the NFMI transceiver device 102 communicates with at least one user device 106 directly. In some embodiments, the NFMI transceiver device 102 communicates with at least one user device 106 through a network such as, for example, a personal area network (PAN), local area network (LAN), metropolitan area network (MAN), wide area network (WAN), cellular network, the Internet, or any combination thereof. In some embodiments, the NFMI transceiver device 102 communicates to a user device 106 through another user device 106. For example, the NFMI transceiver device 102 may communicate (using, for example, Bluetooth™ or NFC) with a patient's mobile phone (acting as a user device 106) and the patient's mobile phone may communicate (using, for example, cellular communications or WiFi over the Internet or other network or combination of networks) with a server or computer (acting as another user device 106) at a healthcare facility (such as a hospital, clinic, or doctor's office).

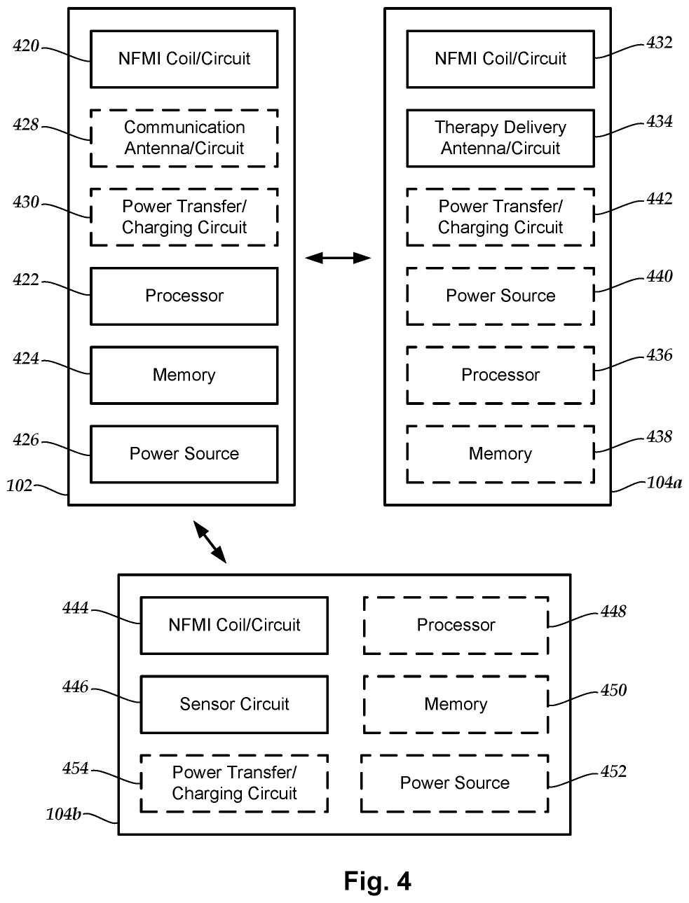

FIG. 4 is a functional block diagram of one embodiment of a NFMI transceiver device 102, one embodiment of a therapy delivery device 104a, and one embodiment of a sensor device 104b. Other embodiments of these devices may include more or fewer components than those illustrated in FIG. 4.

The NFMI transceiver device 102 includes a NFMI coil/transceiver circuit 420 (i.e., a NFMI transceiver), a processor 422, a memory 424, a power source 426 (for example, a battery), an optional communications antenna/circuit 428 for communication with a user device 106 (FIG. 1), and an optional power transfer/charging circuit 430 for transferring power to a therapy delivery device 104a or sensor device 104b. In some embodiments, the optional communications antenna/circuit 428 provides for communication to a user device 106 (FIG. 1) and can be selected from any suitable communications technique including, but not limited to, Bluetooth™, near field communications (NFC), wireless fidelity (WiFi), satellite communication, cellular communication, Infrared Data Association standard (IrDA), or the like or any combination thereof. In some embodiments, the NFMI transceiver device 102 may communicate with a user device 106 using NFMI.

The therapy delivery device 104a includes a NFMI coil/transceiver circuit 432 (i.e., a NFMI transceiver) or, alternatively, a NFMI coil/receiver circuit (i.e., a NFMI receiver), a therapy delivery circuit 434, an optional processor 436, an optional memory 438, an optional power source 440 (for example, a battery), and an optional power transfer/charging circuit 442 for receiving power from the NFMI transceiver device 102 or other power source. The NFMI coil/transceiver circuit 432 (or NFMI coil/receiver circuit) receives signals from the NFMI transceiver device.

The sensor device 104b includes a NFMI coil/transceiver circuit 444 (i.e., a NFMI transceiver) or, alternatively, a NFMI coil/transmitter circuit (i.e., a NFMI transmitter), a sensor circuit 446, an optional processor 448, an optional memory 450, an optional power source 452 (for example, a battery), and an optional power transfer/charging circuit 454 for receiving power from the NFMI transceiver device 102 or other power source. The sensor circuit 446 of the sensor device 104b will depend on the type of sensor that is used. Examples of types of sensors are listed above. The sensor circuit 446 produces sensor signals based on observation of the patient. These sensor signals may be raw output of the sensor or may be processed (for example, using the processor 448 or other processing circuitry) to produce modified output of the sensor or even data based on the raw output of the sensor. The NFMI coil/transceiver circuit 444 (or NFMI coil/transmitter circuit) transmits the sensor signals to the NFMI transceiver device.

The NFMI coil/transceiver circuit 420, 432, 444 include a coil 360 (FIG. 3) and associated circuitry for transmitting or receiving (or both transmitting and receiving) a signal using magnetic induction to/from a NFMI transceiver device, a therapy delivery device 104a, a sensor device 104b, or a user device 106 (FIG. 1). Any suitable coil and transceiver (or transmitter or receiver) circuit can be used. Examples of coils and transceiver circuits for NFMI transmitting and receiving are known. Examples of NFMI devices include, but are not limited to, those described in U.S. Pat. Nos. 8,838,022; 8,929,809; 9,300,367; 9,455,771; 9,742,471; and 10,015,623, all of which are incorporated herein by reference in their entireties.

The power source 426, 440, 452 can be any suitable power source including, but not limited to, batteries, power cells, or the like or any combination thereof. In at least some embodiments, the power source is rechargeable. In at least some embodiments, the NFMI transceiver device 102 includes a power transfer/charging circuit 430 that can be used to charge a power source 440, 452 in the therapy delivery device 104a or sensor device 104b. In at least some embodiments, the power transfer/charging circuit 430 may utilize coil of the NFMI coil/transceiver circuit 420 to deliver power to the therapy delivery device 104a or sensor device 104b. In at least some embodiments, the NFMI coil/transceiver circuit 432, 444 of the therapy delivery device 104a or sensor device 104b can receive the power and deliver to the power transfer/charging circuit 442, 454. In other embodiments, a separate antenna or coil in the power transfer/charging circuit 430 may be used to deliver the power to the power transfer/charging circuit 442, 454 of the therapy delivery device 104a or sensor device 104b for charging the power source 440, 452. In at least some embodiments, a separate charger (not shown) may be used to the charge the power source 440, 452 in the therapy delivery device 104a or sensor device 104b. In at least some embodiments, the power source 426 of the NFMI transceiver 102 may be charged wirelessly or through a wired connection (for example, by attaching a charging cord to a charging port of the NFMI transceiver).

In other embodiments, a therapy delivery device 104a or sensor device 104b may not have a dedicated power source and the NFMI transceiver device 102 (or other device) may deliver power for operation of the therapy delivery device or sensor through the power transfer/charging circuits 430,442, 454 which may utilize the NFMI coil/transceiver circuits 420, 432, 444.

Any suitable processor 422, 436, 448 can be used including, but not limited to, microprocessors, application specific integrated circuits (ASICs), other integrated circuits, or the like or any combination thereof. Any suitable memory 422, 438, 450 can be used including, but not limited to, RAM, ROM, EEPROM, flash memory, or the like or any combination thereof.

The processor 436 can be optional in the therapy delivery device 104a. For example, a processor 436 may be optional if the NFMI signal received by the therapy delivery device 104a produces the desired therapy signal in the therapy delivery circuit 434. In other embodiments, the processor 436 in the therapy delivery device 104a can be programmed or otherwise operated using the NFMI signal from the NFMI transceiver device 102 to deliver, or modify delivery of, therapy using the therapy delivery circuit 434. For example, the NFMI signal from the NFMI transceiver device 102 may include new or updated parameters for therapy delivery, initiate therapy delivery, halt therapy delivery, or the like or any combination thereof. Examples of parameters for therapy delivery include, but are not limited to, amplitude, frequency, or, if pulsed, pulsewidth, pulse duration, or pulse parameter.

The processor 448 may be optional in the sensor device 104b. For example, a processor 448 may be optional if the signal from the sensor circuit 446 can be transmitted to the NFMI transceiver 102 for processing. In other embodiments, the processor 448 in the sensor device 104b can be programmed or otherwise operated using the NFMI signal from the NFMI transceiver device 102 to operate, or modify operation of, the sensor 105b. The processor 448 may also process, partially or fully, signals from the sensor circuit 446 to produce data or signals that are transmitted to the NFMI transceiver device 102. Other processors in the NFMI transceiver device 102 or the user device 106 (or other devices) may fully or partially process data or signals transmitted form the sensor device 104b.

The therapy delivery device 104b includes a therapy delivery circuit 434. Any suitable therapy delivery circuit 434 can be used. In at least some embodiments, at least one of the therapy delivery devices 104b has a therapy delivery circuit that generates ultra-low radio frequency energy. In at least some embodiments, the delivery of ultra-low radio frequency energy can be a therapeutic delivery. In at least some embodiments, the therapeutic delivery can be for the treatment of cancer or other diseases or disorder.

Examples of therapy delivery using ultra-low radio frequency energy can be found in U.S. Pat. Nos. 6,724,188; 6,952,652; 6,995,558; 7,081,747; 7,412,340; 10,046,172 and 9,417,257; U.S. Patent Application Publications Nos. 2019/0143135 and 2019/0184188; and PCT Patent Application Publication WO 2019/070911, all of which are incorporated herein by reference in their entireties. In at least some embodiments, the delivery of ultra-low radio frequency energy includes the generation of a magnetic field having a field strength of up to 1 Gauss. In at least some embodiments, the delivery of ultra-low radio frequency energy includes the generation of a therapeutic magnetic signal having a frequency in the range of 0.1 Hz to 22 kHz or in the range of 1 Hz to 22 kHz.

The therapy delivery circuit 434 can include, for example, a signal generator 535 (FIG. 5) to produce a therapeutic magnetic signal, such as a therapeutic signal for ultra-low radio frequency energy. The therapy delivery circuit 434 can also include a transducer 537 (FIG. 5), for example, a coil or antenna, to deliver the therapeutic magnetic signal to the patient.

In at least some embodiments, the ultra-low radio frequency energy therapy is based on the measurement of a signal generated using a target molecule, for example, a magnetic field generated by a solvated target molecule. Such measurement may include, for example, injecting noise into the sample in the container and recording the resulting magnetic field, as described in the references cited above. Although not limited to a particular hypothesis or mechanism of action, it is thought that the measurements may capture features of a recorded target molecule that can alter behavior of cells, proteins, or other biological agents. Electron and charge transfer are central to many biological processes and are a direct result of interacting surface potentials. Although not limited to a particular hypothesis or mechanism of action, artificial magnetic fields may be capable of triggering a receptor response and conformational change in the absence of a physical drug or molecular agonist.

Superconducting quantum interference device (SQUID) based technology has been used for these measurements. In at least some embodiments, the unique and specific ultra-low radio frequency energy can be used to induce electron and charge transfer in a defined bioactive target, altering cell dynamics to produce a therapeutic response. In at least some embodiments, to provide therapy, a ultra-low radio frequency energy cognate is delivered locally and non-systemically via a medical device. Pre-clinical and clinical studies suggest that ultra-low radio frequency energy therapy provides the ability to specifically regulate metabolic pathways and replicate known mechanisms of action for proven commercial drugs.

Examples of affecting biologic activity with ultra-low radio frequency energy fields includes experiments conducted to demonstrate the specificity and cellular effects of a specific ultra-low radio frequency energy targeting epidermal growth factor receptor, EGFR, on glioblastoma cell line U-87 MG. At 48 and 72 hrs, EGFR inhibition by the ultra-low radio frequency energy reduced the level of EGFR protein by 27% and 73%, respectively. These data indicate that ultra-low radio frequency energy can inhibit gene expression at the transcriptional and protein levels, similar to what is observed with physical small interfering RNA (siRNA) inhibition. Specific EGFR knockdown effect was detected in U-87 MG cells treated with ultra-low radio frequency energy using an 80 gene PCR-based array. See, “Effects of Magnetic Fields on Biological Systems An Overview”; X. Figueroa, Y. Green, D. M. Murray, and M. Butters; EMulate Therapeutics; Mar. 6, 2020.

In another example, ultra-low radio frequency energy therapy was provided as a cancer treatment for over 400 dogs (pets) with naturally occurring malignancies. Interim review of the first 200 pets observed partial responses and complete responses in over 20 different tumor types. No clinically important or significant toxicities (Grade 3 or 4) were observed.

FIGS. 5A and 5B illustrate one embodiment of a therapy delivery device 504a that includes a NFMI coil/transceiver circuit 532, a therapy delivery circuit 534 for delivery of a magnetic therapeutic signal such as a ultra-low radio frequency energy signal, a battery 540 (as a power source), and an optional charging circuit 542. These elements of the therapy delivery device 504a are disposed on a substrate 556 which can be, for example, a flex circuit substrate or any other suitable flexible substrate. In some embodiments, the substrate 556 can include an adhesive 555 disposed on a back surface 557 of the substrate to adhere the therapy delivery device 504a to the skin of the patient, similar to an adhesive bandage. In at least some embodiments, the therapy delivery device 504a may be otherwise attached to the skin of the patient (e.g., using tape or a bandage) or worn by the patient at or near the treatment site. Optionally, a top substrate 558 can be disposed over the substrate 556 and the components listed above to provide protection to those components.

FIG. 6 illustrates one embodiment of a NFMI transceiver device 602 with some optional features such as a display 670, one or more buttons 672 or other input devices (such as a keyboard), and one or more lights 674. In at least some embodiments, the display 670 may display instructions or information about operation or warnings or the like or any combination thereof. In at least some embodiments, the lights 674 may indicate that the NFMI transceiver device is on/off 102, the status of system devices 104, the status of a power source, warnings about low power source or loss of signal or the like or any combination thereof.

In at least some embodiments, the one or more buttons 672 or other input devices may be used by the patient or other user to direct delivery of therapy or alter therapy parameters or the like or any combination thereof. In at least some embodiments, the delivery of therapy or the alteration of therapy parameters may be restricted to a user with credentials, such as a password or other identification.

In at least some embodiments, the use of NFMI for communication between the NFMI transceiver device and the system devices can enable wireless (e.g., cable free) wearable therapy delivery devices for ultra-low radio frequency energy. In at least some embodiments, the use of NFMI for communication between the NFMI transceiver device and the system devices can enable real-time communication with wearable and implantable therapy delivery devices for ultra-low radio frequency energy. In at least some embodiments, the use of NFMI for communication between the NFMI transceiver device and the system devices can enable relatively low attenuation of communication (into the body of the patient) with implantable therapy delivery devices for ultra-low radio frequency energy.

In at least some embodiments, the use of NFMI for communication between the NFMI transceiver device and the system devices can enable simultaneous operation of a therapy delivery device for ultra-low radio frequency energy and at least one sensor device. In at least some embodiments, the use of NFMI for communication between the NFMI transceiver device and the system devices can enable wireless power transfer to a wearable or implantable therapy delivery device for ultra-low radio frequency energy. In at least some embodiments, the use of NFMI for communication between the NFMI transceiver device and the system devices can enable security of signals between the NFMI transceiver device and the system devices.

In at least some embodiments, the present systems can enable simultaneous communication with a user device using Bluetooth™ or other communication techniques. Such communication may allow a user to control the NFMI transceiver device and one or more therapy delivery devices for ultra-low radio frequency energy (and, optionally, one or more sensor devices.)

The above specification provides a description of the invention and the manufacture and use of the invention. Since many embodiments of the invention can be made without departing from the spirit and scope of the invention, the invention also resides in the claims hereinafter appended.

What is claimed as new and desired to be protected is:

1. A therapy delivery system, comprising:
a near field magnetic induction (NFMI) transceiver device configured to be worn or otherwise disposed on a patient, the NFMI transceiver device comprising a NFMI transceiver; and
a therapy delivery device comprising
a NFMI receiver configured to receive signals from the NFMI transceiver device, and
a therapy delivery circuit configured to deliver a therapeutic magnetic signal, based, at least in part, on the received signals from the NFMI transceiver device, to the patient when the patient wears the therapy delivery device or has the therapy delivery device disposed on skin of the patient or has the therapy delivery device implanted.

2. The therapy delivery system of claim 1, wherein the therapy delivery device further comprises a power source coupled to the NFMI receiver and the therapy delivery circuit.

3. The therapy delivery system of claim 2, wherein the therapy delivery device further comprises a charging circuit coupled to the power source and configured to receive power from an external source to charge the power source.

4. The therapy delivery system of claim 3, wherein the NFMI transceiver device comprises a charging circuit configured to provide power to the charging circuit of the therapy delivery device.

5. The therapy delivery system of claim 4, wherein the charging circuit of the NFMI transceiver device is configured to utilize the NFMI transceiver to provide the power to the charging circuit of the therapy delivery device.

6. The therapy delivery system of claim 1, wherein the therapy delivery circuit is configured to deliver the therapeutic magnetic signal having a frequency in a range of 0.001 Hz to 22 kHz.

7. The therapy delivery system of claim 1, wherein the therapy delivery device further comprises a first substrate and the therapy delivery circuit and the NFMI receiver are disposed on the first substrate.

8. The therapy delivery system of claim 7, wherein the therapy delivery device further comprises an adhesive disposed on a surface of the substrate for adhering the therapy delivery device to the skin of the patient.

9. The therapy delivery system of claim 7, wherein the therapy delivery device comprises a second substrate disposed over the therapy delivery circuit and the NFMI receiver and attached to the first substrate.

10. The therapy delivery system of claim 1, further comprising a sensor device comprising
a sensor circuit configured to produce a sensor signal based on observation of the patient by the sensor device; and
a NFMI transmitter configured to transmit the sensor signal to the NFMI transceiver device.

11. The therapy delivery system of claim 10, wherein at least one of the therapy delivery device or the sensor device is implantable in the patient.

12. The therapy delivery system of claim 1, wherein the NFMI transceiver device further comprises a communications circuit for communication, other than NFMI, to a user device.

13. The therapy delivery system of claim 12, wherein the therapy delivery system further comprises the user device configured to communicate with the NFMI transceiver device through the communications circuit of the NFMI transceiver device.

14. The therapy delivery system of claim 13, wherein the user device comprises a mobile phone, a tablet, or a computer.

15. The therapy delivery system of claim 1, wherein the NFMI transceiver device further comprises a processor coupled the NFMI transceiver and a memory coupled to the processor.

16. A method of delivering therapy to patient, the method comprising:
receiving, at a therapy delivery device, a near field magnetic induction (NFMI) signal from a NFMI transceiver device worn or otherwise disposed on the patient; and
in response to the received NFMI signal, delivering a therapeutic magnetic signal to the patient using the therapy delivery device, wherein the therapy delivery device is worn by the patient, disposed on skin of the patient, or implanted in the patient.

17. The method of claim 16, further comprising receiving, at the therapy delivery device, a charging signal from the NFMI transceiver device to charge a power source of the therapy delivery device.

18. The method of claim 17, wherein receiving the charging signal comprises receiving the charging signal using NFMI.

19. The method of claim 16, further comprising receiving, at the NFMI transceiver device, a NFMI signal from a sensor device that is worn by the patient, disposed on skin of the patient, or implanted in the patient.

20. The method of claim 16, further comprising communicating, from the NFMI transceiver device without using NFMI, with a user device.
